(19) United States
(12) Patent Application Publication
(10) Pub. No.: US 2016/0331709 A1
(43) Pub. Date: Nov. 17, 2016

(54) Balancing an Unbalanced Sympathetic Nervous System

(71) Applicant: Alicia Marie Wiggins, Woodbury, MN (US)

(72) Inventor: Alicia Marie Wiggins, Woodbury, MN (US)

(73) Assignee: WOW LLC???

(21) Appl. No.: 15/152,548

(22) Filed: May 12, 2016

Related U.S. Application Data
(60) Provisional application No. 62/161,120, filed on May 13, 2015.

(51) Int. Cl. A61K 31/198; A61K 31/593; A61K 31/675; A61K 38/02; A61K 31/191

(52) U.S. Cl. CPC A61K 31/198; A61K 38/02; A61K 31/191; A61K 31/675; A61K 31/593

(57) ABSTRACT

Many illnesses generally result from chemical imbalances and deficiencies in the Sympathetic Nervous System. The composition of the invention provides the body with targeted amino acids that manage the Sympathetic Nervous system to facilitate neuroregneration at the cellular level. This is done with little or no adverse side effects to the body and can be taken with other pharmaceuticals and nutritional supplements without adverse interactions with them.

### FIELD OF THE INVENTION

The present invention relates to a compound that improves an imbalanced sympathetic nervous system and method of using same.

### BACKGROUND OF THE INVENTION

There is a need for a more medicinally effective way to treat chronic pain, mental health, immune issues, and addiction than is currently available.

Many people suffer from chronic pain, mental health issues such as stress and depression, addictions, and matters of immune health. Currently, efforts to treat these conditions rely on pharmaceuticals and herbal supplements. Both have mixed results with frequent adverse side effects or adverse interactions with pharmaceuticals or herbal supplements.

There is still a need for a more medicinally effective treatment of these conditions with less adverse side effects or adverse interactions with other pharmaceuticals or herbal supplements.

### SUMMARY OF THE INVENTION

Many illnesses generally result from chemical imbalances and deficiencies in the sympathetic nervous system. The composition of the invention provides the body with targeted amino acids that manage the Sympathetic Nervous system to facilitate neuroregeneration at the cellular level. This is done with little or no adverse side effects to the body and can be taken with other pharmaceuticals and nutritional supplements without adverse interactions with them. The invention comprises two aspects, a composition and a method of use.

v. The composition aspect of the invention for achieving a greater balanced in an imbalanced sympathetic nervous system comprises a medicinally effective amount of three amino acids. These are DL-Phenylalanine, L-Tyrosine, and L-Glutamine. The composition of the invention also does not include a discernibly effective amount of each of four non-amino acid materials or additional amino acids in various forms. These six materials are St. John's Wort, chromium picolinate, digestive enzymes such as pepsin and bromelain, furanones, branched chain amino acids, and peptides containing more than three peptide forms of amino acids different from those of DL-Phenylalanine, L-Tyrosine, and L-Glutamine.

The method aspect of the invention for achieving a greater balance in an imbalanced sympathetic nervous system comprises two steps. The first step comprises providing a composition as described above. The second step comprises taking a recommended quantity of the composition over a recommended time frame to reduce symptoms.

Benefits of the invention relate to the health of the body. This invention produces beneficial effects to people suffering from issues of chronic pain, mental health, immune issues, and addiction that are currently difficult to treat effectively. In addition, these beneficial effects are achieved with little or no adverse side effects to the body or adverse interactions with pharmaceuticals, and nutritional supplements that may also be taken by a person. The invention also has beneficial effects for the treatment of mammals such as dogs and cats that may be suffering from issues such as separation anxiety and chronic pain.

As used herein:

“Adverse effective amount” means a quantity that has a discernible adverse effect such as nausea, headache, or diarrhea.

“Discernibly effective amount” means an amount sufficient to cause a noticeable effect on the body or mind of a mammal.

“Medicinally effective amount” is an amount shown to significantly reduce symptoms of a mammal suffering from pain, moods, immune, and addiction, and does not significantly result in additional symptoms or illnesses not currently experienced.

### DETAILED DESCRIPTION OF SOME EMBODIMENTS OF THE INVENTION

This invention is a composition that more effectively addresses chronic pain, moods or mental health conditions such as depression, addiction, and immune issues than are presently addressed by traditional treatment methods. The composition of the invention has minimal adverse side effects to the body, and minimal adverse side reactions with other compounds such as pharmaceuticals and herbal supplements.

### My Journey

As a person with chronic illnesses since age 20, and various maladies, along with the ineffectiveness of traditional medicine, I felt the need to research my own and my family's health issues.

I initially had five herniated disks and the chronic pain associated with that. The side effects of the medication given for pain affected my moods and my focus. I developed allergic and other adverse reactions to those medications and experienced illnesses including but not limited to chronic infections. I was on antibiotics every 4 to 6 weeks for 25 years for recurring sinus and ear infections. The repeated use of antibiotics caused Candida yeast issues, eventually leaky gut, food, and chemical intolerances. I also had pneumonia 2 to 3 times a year, I was eventually diagnosed with Severe

Environmental illness, mold spores were growing in my brain and lungs, and I received three allergy shots per week, and a diagnosis of Asthma. Many of these illnesses are associated with an overtaxed and biochemically deficient Immune system. Over time, a diagnosis of Fibromyalgia, Polycystic ovarian syndrome, and mercury amalgam toxicity as well, were further demonstrations of an overtaxed and biochemically deficient immune system.

I learned that the package of illnesses is common among people that are chronically ill and on antibiotics or other treatments for a long time. This is important, because I was so ill with so many diagnoses that my body was not able to respond to traditional medical treatments because of adverse interactions and side effects. My body had side effects, or poor response to every medication or injection I had been given. I had been to world-renowned doctors and clinics with limited or no success.

Because none of my suffering was being healed or well managed by existing drugs or treatments, I was led to search for alternative courses of treatment. I discovered that there are many shared symptoms among chronic illnesses. These all led back to the

Sympathetic Nervous System. At that time, I had been exploring herbal medications and became disappointed in their many side effects along with interactions with other herbals, nutritional supplements, foods, and pharmaceuticals. It is known that herbals have negative side effects with over 50% of pharmaceuticals on the market today.

In some respects, the causes of many of my illnesses were complex. Because of my many sensitivities and because of frequent adverse side effects of medications that I took, and the medications that are then used to treat those side effects, and the adverse side effects of those medications that I took to treat those side effects, and the adverse interactions between the medications that I took, any beneficial effect of one was often compromised and frequently eliminated.

Eventually, I lost my job in 2006 due to my chronic health issues and adverse interactions that had spiraled down to a point where I was unable to effectively function in a work environment. That year it was discovered that I had a mild traumatic brain injury, with depression, migraines, and bipolar tendencies.

### Neurobioceuticals

I began to study biochemical nutrition and orthomolecular medicine nearly 30 years ago in an effort to find a more effective solution to my consistently declining health and managing my chronic pain from five herniated disks and the resulting complications as discussed above.

### Biochemical Research

It is known that we are made of water and amino acids. Vitamins and minerals comprise less than 2% of our structure. These statements directly correlated with the work that I had studied in orthomolecular medicine from scientists, each of whose life's work was devoted to healing. Some of these scientists were Nobel Prize winners and Nobel Prize nominees. The body of research came to include the National Institute of Health (NIH), as well as the Journal of American Medicine, the British Medical Journals, and the New England Journal of Medicine, even including 175 volumes of research published by the United States Army entitled “The Effects of Nutrition on the United States Soldier”. The foundation of orthomolecular medicine is that illness and disease come as a result of long-term deficiency of one or more nutrients in the field of vitamins and minerals. However, I found that because vitamins and minerals comprised less than 2% of the body, I suspected that further investigation into vitamins and minerals would be ineffective in dealing with my issues. However, I believed applying the principles of orthomolecular medicine to amino acids that comprise most of the body would be more fruitful in my efforts to manage my illnesses as a result of the five herniated disks and chronic illness as discussed above.

Neuroregenerative medicine is the regeneration of health through the treatment of deficiencies of biochemicals normally made by or ingested by the body. These treatments typically consist of the addition of those biochemicals. However, the long term healing of this approach has its limitations because this treatment consists of the injecting of specific biochemicals to treat targeted ailments for a physiological effect and not a biochemical effect.

I combined aspects of the above teachings into a hybrid form of treatment that I call Neurobioceuticals. I used the teaching that of Orthomolecular medicine that illness and disease is founded in nutritional deficiencies of one or more nutrients. However, I applied this to amino acids instead of vitamins and minerals because amino acids comprise most of the body, then I took the addition of targeted biochemicals that treat specific physiological damage in the body and applied amino acids to treat biochemical deficiencies in the body. I used the principles taught in Neuroregeneration to achieve healing but selected the use of amino acids instead of biochemicals because they comprise most of the body. An amino acid may treat a dozen biochemicals to aid in healing whereas the work in Neuroregenerative medicine addresses it by adding only one or a few biochemicals to the body to treat a targeted physiological response. Amino acids generate numerous biochemicals that have numerous benefits on the body, and picking one biochemical to treat one physiological malady is believed by me to not lead to the long-term healthy outcome, that I was seeking. Physiological maladies have an adverse biochemical effect on the body. For example, I discovered that long term chronic pain such as I experience with my herniated disks, causes the use of specific biochemicals being used to fight or manage the pain. This creates a deficiency of specific biochemicals within the body that are used for other things as well. I concluded that adding a deficient single or just a few selected biochemicals has a narrow targeted effectiveness in long term healing. In contrast adding the deficient amino acids to the body allows it to function as intended facilitating the creation of multiple biochemicals that the body needs for multiple uses, leading to a broader effectiveness in the recovery and management of health.

### Problem/Solution

People have chronic pain, mood, immune issues, and addiction problems that are not being addressed satisfactorily via current methods. Pharmaceuticals provide limited and short-term results with frequent adverse reactions and side effects. Herbs also provide limited results with adverse interactions and side effects with other foods, nutrients and pharmaceuticals. There is a need for a composition that provides long-term results for treating the Sympathetic Nervous System directly which I learned manages pain, moods, the immune system and addiction with minimal to no side effects with pharmaceuticals, herbal remedies, and nutritional supplements.

### The Central Nervous System

The central nervous system (CNS) is the part of the nervous system consisting of the brain and spinal cord. The central nervous system is so named because it integrates information it receives from, and coordinates and influences the activity of, all parts of the bodies. The human nervous system is divided into the central nervous system (CNS) and the peripheral nervous system (PNS). The CNS, in turn, is divided into the brain and the spinal cord, which lie in the cranial cavity of the skull and the vertebral canal, respectively. The CNS and the PNS, acting in concert, integrate sensory information and control motor and cognitive functions.

### The Autonomic Nervous System

The autonomic nervous system (ANS), also known as the visceral nervous system and involuntary nervous system, is a division of the peripheral nervous system that influences the function of internal organs. The autonomic nervous system is a control system that acts largely unconsciously and regulates the heart rate, digestion, respiratory rate, pupillary response, urination, and sexual arousal. This system is the primary mechanism in control of the fight-or-flight response and its role is mediated by two different components, the Sympathetic Nervous System and the Parasympathetic Nervous System.

### The Parasympathetic Nervous System

The autonomic nervous system is responsible for regulating the body's unconscious actions. The parasympathetic system is responsible for stimulation of “rest-and-digest” or “feed and breed” activities that occur when the body is at rest, especially after eating, including sexual arousal, salivation, lacrimation (tears), urination, digestion and defecation. Its action is described as being complementary to that of the sympathetic nervous system, that is responsible for stimulating activities associated with the fight-or-flight response.

### The Sympathetic Nervous System

The Sympathetic Nervous system is often called the “fight or flight” nervous system. It manages Pain, Moods, Immune, and Addiction and is known to be the biochemical center of human health.

Illness and disease lead back to the sympathetic nervous system. The sympathetic nervous system is known to be the biochemical center of health. The sympathetic nervous system works in tandem with the parasympathetic nervous system, and together they make the Autonomic Nervous System.

I believed that I would be able to manage my pain and moods as the sympathetic nervous system manages pain, moods, immune system, and addiction-related maladies if I learned what amino acids created the biochemicals that manage and run the sympathetic nervous system.

I discovered that the sympathetic nervous system is managed by three primary biochemicals. They are dopamine, serotonin, and norepinephrine. These three biochemicals, sometimes called neurotransmitters or neurohormones, also play a vital role in mental health and are frequently associated with brain chemistry for the purpose of managing mental health.

I then explored which amino acids create these important biochemicals. I discovered specific amino acids that create these biochemicals. These amino acids also interact with one another making them vastly more medicinally effective in creating dopamine, serotonin, and norepinephrine as mentioned above. I also discovered that there were few if any side effects associated with the ingestion of these amino acids.

### Current Products

Amino acids are available in single or some combinations of other amino acids, vitamins, and herbs such as for weight loss. They have minimal result based upon formulations with minimal medicinally effective dosages or cause negative side effects.

### My Initial Experiments

I performed various trials during my course of discovering what might effectively treat my ailments without causing additional ones.

### Case 1

My initial experiment was with a man with a crack cocaine addiction for approximately five years. He had been through chemical dependency treatment numerous times and was unable to overcome his addiction. He used the product for two months. He reported he was free from cravings, had no mood swings as well as being relieved from sugar cravings. After two months, he became complacent about taking the formulation and returned to his previous addiction.

### Case 2

I came to know a single mother in her 30's. She expressed a need for help with her mental illness. She expressed that she was hospitalized for up to six months with mental illness. She also discussed a period of time when she was paralyzed from the neck down and hospitalized for six weeks. She stated to me that during her hospital stay the doctors told her after six weeks of many tests that her physical status was a result of her mental illness status that there was nothing wrong with her body. This woman stated to me that she wanted to be treated with my formulation. She called me within two weeks following the beginning of treatment, stating that she had felt vital, focused, and free of anxiety and that her headaches and digestive issues were improved as well. She took the formulation for a period of three months. During that time, she called me regularly to exclaim the improvement in her mental and physical health.

### Case 3

Eventually I began taking my own custom formulated capsules for my chronic pain and moods. The chronic pain was significantly decreased and moodiness including depression was no longer debilitating.

Surprisingly I discovered that my immune system began to recover as well. I learned this as a result of no longer needing antibiotics and antifungals, the mold spores in my brain were gone the symptoms associated with mold spore growth were no longer there. Upon visiting an allergist, I learned that I no longer had Asthma.

I was very surprised when I came to learn that I no longer had Asthma. Asthma is an incurable disease. Certainly all of the other maladies were possible to treat and in rare cases, cure; but Asthma was incurable, or so we are told.

### Case 4

A young man was diagnosed with bipolar disorder and could not control his behavior. He explained that traditional mental health services had not served him well. I explained to him what this product does, and how it does it, and he was anxious to try it. This young man called me four days into taking the product and told me how he felt a weight lifted off of his shoulders, he had been waking up naturally and refreshed in the morning and was thrilled to be out looking for work. He did not have delusions or visions and was feeling very balanced. He was able to get a job and was doing very well last I spoke to him.

### Case 5

A 16-year old female came to me to help alleviate her depression. She was afraid to take antidepressants because of the effects they had on her. Within two weeks' time following the initiation of treatment, she felt a calm confidence, ability to focus, and make good decisions. She also called me one day to tell me she had finally been able to eat meat again. Apparently, she had been unable to digest anything but noodles for years. Clearly, we see the healing of the gut here as well.

### Effects on Animals

I have come to know of several dogs with arthritis, separation anxiety, behavioral issues, and gut issues who have enjoyed a sprinkle of this product on their food each morning and find great results as well.

### The Invention

Embodiments in accordance with the invention include dietary supplements that are particularly useful in nutrition and sympathetic nervous system balance. In the following description, numerous specific details are set forth such as specific formulations, processing steps, processing parameters, etc. in order to provide a thorough understanding of the invention.

The invention is a dietary supplement composition comprising in one embodiment, three specific amino acids, minerals, and vitamins. The composition is developed particularly for persons who desire recovery of the sympathetic nervous system. It is important that additions of other materials including other amino acids be kept to a minimum to achieve medicinally effective amounts of the amino acids in the composition of the invention

Studies reveal that biochemical deficiencies affect a network of functions in mammalian body and brain. Overstress of life can be shown to cause these “network” deficiencies. I have found that to medicinally effectively treat an imbalanced Sympathetic Nervous System requires a number of essential compounds and elements. The composition of the invention supplies the full range of these needed compounds. The amino acid components supply or convert to the biochemicals required by the Sympathetic Nervous System. In contrast, vitamins assist in metabolism. The mineral in the composition interacts with over 800 processes in the body as well as other ingredients act as neurotransmitters between the hypothalamus and the central nervous system, which regulate all the body's autonomous functions including the balance of the Sympathetic Nervous System.

The network deficiencies of amino acids, vitamins, and minerals recognized by this inventor are so common that a majority of people in the general population are likely deficient to some degree. It is therefore not surprising that a significant portion of mammals are suffering symptoms of a deficient/imbalanced sympathetic nervous system, and that their efforts to return to full health through traditional medical or other alternative medical intervention are often unsuccessful.

The active ingredients in the embodiment are three amino acids that are known to function as neurotransmitters: (1) DL-Phenylalanine, (2) L-Tyrosine, and (3) L-Glutamine in an amount of 50-65 percent by weight of the total composition. Medicinally effective are based upon the amounts. It is important not to dilute the amounts below the medicinally effective amount, due to using too many of the amino acids, or using too much of additional amino acids. The individual components are described.

DL-Phenylalanine (DLPA) is a combination of D-Phenylalanine and L-Phenylalanine and is an essential amino acid that is a neurotransmitter. The D-Phenylalanine component when used alone has been reported to alleviate chronic pain. However, it is common to experience overt nausea. The L-Phenylalanine component, when used alone has serious side effects, including the creation of manic episodes in those with a bipolar diagnosis as well as further complicating the issues of Attention Deficit Disorder and Attention Deficit Hyperactivity Disorder. When used in combination DL-Phenylalanine side effects are minimal except in the situation where a person is born with the diagnosis of Phenylketonuria which is a rare genetic disorder diagnosed at birth. The DL version is superior because it negates the potential for side effects mentioned above with problems associated with D or L alone. Some of DLPA's main functions are improvements in memory and mental alertness and depression alleviation.

L-Tyrosine is an amino acid and neurotransmitter that stimulates and modifies brain activity and supplies the precursor of the thyroid gland. Clinical studies show that L-Tyrosine helps control depression and anxiety. In contrast, N-Acetyl L-Tyrosine is not preferred because there is not enough research to support its use at present.

L-Glutamine is a natural form of glutamic acid and is the most abundant amino acid in the body and brain and decreases fatigue and depression. L-Glutamine is a high-ranking neurotransmitter and with an important role in stimulating and modifying brain activity. Clinical studies have shown that L-Glutamine supplementation also controls depression and anxiety and is essential for healing of the gut.

As suggested earlier, additional amino acids should be kept at a minimum. Too many amino acids, when used in a formulation, or in an N-Acetyl form or in a peptide chain reduce efficacy due to insufficient amounts of the above-mentioned amino acids of the invention. This is because certain amino acids compete with each other in the digestive process to cross the blood brain barrier. Trace amounts can be ineffective or merely neuroprotective. However, our formulation not only is neuroprotective, but has medicinally effective amounts to return balance to the Sympathetic Nervous System. Therefore it has neuroregenerative properties as well as neuroprotective properties.

Also, the addition of minerals and vitamins has been found to enhance performance of the composition of the invention. In particular, three have been found to have particularly beneficial effects and may be used individually or in combination without any adverse effects. The three of particular interest are Magnesium Gluconate, Vitamin B6, also known as P5P or Pyridoxal-phosphate, and D3 also known as Cholecalciferol.

Magnesium Gluconate is a medicinally effective way of introducing Magnesium into the body. Magnesium gluconate is a compound with formula MgC₁₂H₂₂O₁₄. It is the magnesium salt of gluconic acid. Magnesium is an anti-stress mineral. Magnesium effects over 800 processes in the body and plays an important role in making amino acids more bioavailable to balance the sympathetic nervous system, and the brain chemicals dopamine, serotonin, and norepinephrine that are created by the amino acids of the invention. Magnesium can aid in fighting depression, promote a healthy cardiovascular system, keep teeth and bones healthier, and help prevent calcium deposits, kidney stones, and gallstones. One embodiment provides at least this addition.

Vitamin B6 acts as a catalyst in over 100 biochemical reactions in the nervous system and is used in the production of antibodies and red blood cells. This vitamin properly assimilates protein and can also help prevent numerous nervous and skin disorders. The body excretes any excess B vitamins and thus does not store them in the body. Vitamin B6 as described above must be replaced daily to maintain good health. One embodiment provides at least this addition.

Vitamin D3 (cholecalciferol) has been found to affect over 300 genes and is a vitamin derived from 7-dehyrocholesterol. However, Vitamin D3 acquires hormone-like actions when cholecalciferol (Vitamin D3) is converted to 1,25-dihydroxy (Calcitriol) by the liver and kidneys. As a hormone, Calcitriol controls phosphorus, calcium, and bone metabolism and neuromuscular function and is considered to be both a vitamin and a hormone and plays not only a key role in managing winter sadness but also in moods throughout the year. D3 has been linked to cancer prevention (reducing risk by 30-50%), assisting with calcium balance, thyroid function, bone health, headaches, menopause, Multiple Sclerosis, Alzheimer's, Parkinson's, asthma, autism, bipolar, ADD/ADHD, and various mental health diagnoses.

Vitamin D3 deficiency is linked to a surprising number of other health conditions such as depression, back pain, cancer, both insulin resistance and pre-eclampsia during pregnancy, impaired immunity and macular degeneration, and has been implicated in psychiatric and neurologic disorders. Low Vitamin D3 levels play in the development of chronic low back pain in women. Long-established benefits of Vitamin D3 are its ability to improve bone health and the health of the musculoskeletal system. It is well documented that Vitamin D3 deficiency causes osteopenia, precipitates and exacerbates osteoporosis, causes a painful bone disease known as osteomalacia, and exacerbates muscle weakness, which increases the risk of falls and fractures. Vitamin D3 insufficiency may alter the regulatory mechanisms of parathyroid hormone (PTH) and cause a secondary hyperparathyroidism that increases the risk of osteoporosis and fractures. In a recent study, Vitamin D3 deficient subjects scored worse on mental function tests compared to individuals who had higher levels of the Vitamin. Imbalanced circadian rhythm results in daytime sleepiness. A growing number of researchers who have widely studied Vitamin D3 are almost begging the general public to consume more of this important nutrient due to Vitamin D3's high safety profile in doses up to 10,000 IU per day and because of the wide role it plays in our health. In conclusion, Vitamin D3 is in our catalyst group because it consistently demonstrates efficacy in scientific studies as it relates to the sympathethic nervous system (pain moods immune and addiction). One embodiment provides at least this addition.

More than one of the catalysts in the composition may be used together. The composition according to one embodiment of the invention uses teamwork or synergism for maximum results on balancing the sympathetic nervous system and therefore mental health/well being and physical health. The synergism produces results that were heretofore unachievable in prior art. This is because prior art supplements used only one component to address one area (for example) and did not consider managing the sympathetic nervous system overall, allowing recovery from many health maladies and diseases.

Thus for the first time, a composition according to one embodiment of the invention, has been developed that combines amino acids, vitamins, and a mineral to balance the Sympathetic Nervous System and promote metabolic and cellular neuroregeneration performance, brain health, mental/health, wellbeing and physical health.

In addition to many pharmaceuticals often having adverse reactions with other pharmaceuticals, herbs, and some nutrients including minerals and enzymes can have adverse reactions with other foods, pharmaceuticals, and nutrients. In particular, the composition of the invention does not contain three specific materials that have no beneficial effect from an Orthomolecular/Neuroregenerative perspective in addressing the imbalanced sympathetic nervous system, and have adverse effects on the body. These materials are the herbal supplement St John's Wort, chromium picolinate, and digestive enzymes such as pepsin and bromelain.

Discernibly effective amounts of St. John's Wort may have discernable effects on depression but also can have negative side effects with various nutritional supplements, foods, and is reported to interfere with the metabolism of over half of pharmaceutical medicines. St. John's Wort regulates a whole program of genes involved in solubilization and excretion of chemicals from the body. In addition, the CYP3A-PXR mechanism is also triggered by drugs used to treat epilepsy, diabetes, cancer, and inflammation. Thus, there is a situation where a drug can amplify expression of CYP3A, and accelerate the metabolism of other drugs—a recipe for a wide array of drug interactions.

Discernibly effective amounts of Chromium Picolinate may also have adverse effects to the body. Research indicates that chromium picolinate may negatively affect levels of neurotransmitters i.e. substances in the body that transmit nerve impulses. This would result in a further negative impact on an already imbalanced sympathetic nervous system. This may potentially be a concern for people with conditions such as depression, bipolar disorder, and schizophrenia. Chromium picolinate has also been reported to have negative effects if combined with pharmaceuticals for diabetes, and may cause blood glucose levels to dip too low. It is also reported to have adverse reaction with thyroid, mental illness and those who may use steroid medications.

Discernibly effective amounts of Digestive enzymes have no beneficial effect on an imbalanced sympathetic nervous system. Pepsin and Bromelain may also have adverse effects to the body. Pepsin has been reported to cause swelling of the abdomen, disorder of the digestive system, formation of fibrous tissue in the colon, and gallstones. Bromelain has been reported to have severe adverse side effects when used with antibiotics, blood thinners, and those with latex and wheat allergies.

In addition, the invention has no effective amount of furanones. Furanones are a smell enhancement chemical commonly used for foodstuffs, perfumes and cosmetics for both humans and animals to simulate desirable odors. Furanones have little if any effect on the Sympathetic Nervous System. In addition, an odor to a dietary supplement is generally adverse to consumers.

### Medicinally Effective Amounts of Components

The effective amount of the composition of the invention comprises the medicinally effective amount of the combination of the three amino acid components DLPA (DL-Phenylalanine) L-Tyrosine and L-Glutamine. The amino acids have a synergistic relationship that is much less effective if used in alone or in pairs.

In the composition of the invention, each of the components of the invention has a medicinally effective amount to beneficially treat a mammal, typically the medicinally effective amount is similar for a mammal such as, for example, a human, domesticated animals such as dogs and cats, and livestock such as cows and horses. Livestock and domesticated animals tend to have up to four times the upper limit in comparison to a human due to difference in metabolic rate between animals and humans. Because these materials are naturally found in mammals, there are little if any side effects or negative interactions and therefore efficacy tests are not necessary. There is little if any need for testing of these materials in mammals to show if there are adverse side effects in the mammal's body because these materials are innate to the body, broadly researched for toxicity over the past several decades, and are passed as waste if not needed by the body.

A medicinally effective amount is an amount that has either a positive or a negative effect on the body. This is opposed to a merely effective amount that only has a discernible effect on the body by the recipient but does not beneficially or adversely affect the health of the body. The medicinally effective amounts of each of the amino acid components of the invention, DLPA (DL-Phenylalanine) L-Tyrosine and L-Glutamine are between 0.02 mg per pound of body mass per day of the living mammal and 240 mg per pound of body mass per day of the living mammal. This translates to for a 40 lb. dog to between 0.8 mg and 9.6 grams, for a horse or a cow at 1,800 lbs. 36 mg and 432 grams the effective amounts for each of the amino acids for a human is between 0.02 and 180 mgs per pound of body mass per day. This converts to between 2 mg and 18 grams for a 100-pound person and converts to between 4 mg and 36 grams for a 200-pound person. Regarding the non-amino acid components of the invention the medicinally effective amounts of each of Magnesium Gluconate, Vitamin B6 [Pyridoxal-phosphate] are similar to each of the amino acids above. The Vitamin D3 has a medicinally effective amount of 0.0002 mg per pound of body mass per day of mammal to 1 mg per pound of body mass per day of mammal. A similar range is applicable to humans. In formulations, the amino acids should comprise 50 to 90% of the compounds and the non-amino acids should comprise 10-50%.

### Method of Use

An effective form of administering the compound is the composition of the invention can be administered by various ways in various forms as known to the art; these include for example, capsules, tablets, powders, syrups, IV fluids, liquid beverage, and transdermal patches.

In one embodiment of the method of use a human over age 12 years with a need for bringing the Sympathetic Nervous System into balance as discussed above, an effective form of dosing has been found to be an oral administration of 1 capsule twice daily for 2 days, then increase to 2 capsules twice daily for 2 days and then to three capsules twice daily (this is full dose). Dosing times are first thing upon waking and 4-6 hours later. Other dosing regimens may be used depending on the severity of the symptoms of the imbalanced Sympathetic Nervous system.

In the above embodiment the capsule has the following formulation, Vegetarian capsules size 00, with a capacity of 735 mg per capsule, the components for each capsule are 250 mgs of DLPA (DL-Phenylalanine), 250 mgs of L-Tyrosine, 100 mgs of L-Glutamine, 100 mgs of Magnesium Gluconate, 8 mgs of Vitamin B6 (P5P), and 0.04 mgs of Vitamin D3.

Taken on an empty stomach with lightly sweet fruit juice or added to a smoothie. Other means of ingestion may be used depending upon the situation, person, or animal. This regimen is independent of body mass and age of human or mammal.

The regimen is full dose of three capsules twice daily as outlined above for the first year, and then half dose for the second year to create resolution in most cases. In another embodiment where the illnesses are chronic and long term for many years, a maintenance program may be beneficial. Ongoing dose to maintain relief is typically 3 capsules twice daily.

In an embodiment where the human is from birth to 12 years old the composition is dosed based upon weight.

Embodiments of the method of use where the subject is an animal, the dosing is based upon weight and is often greater due to a difference in metabolic rate among animals. Method of delivering dosage for animals is that same as for humans.

Other modifications and changes made to fit particular operating requirements and environments will be apparent to those with ordinary skill in the art. Thus, the invention is not considered limited to the embodiments discussed for purposes of disclosure and covers all changes and modifications that do not constitute departures from the true spirit and scope of this invention.

What is claimed is:

1. A composition for achieving greater balance in an imbalanced sympathetic nervous system, comprising:
a. medicinally effective amount of the combination of:
i. DL-Phenylalanine,
ii. L-Tyrosine, and
iii. L-Glutamine, and
b. no discernibly effective amounts of each of:
i. St. John's Wort,
ii. chromium picolinate,
iii. digestive enzymes such as pepsin and bromelain,
iv. furanones,
v. branched chain amino acids, and
vi. peptides containing more than three peptide forms of amino acids different from those of DL-Phenylalanine, L-Tyrosine and L-Glutamine.

2. The composition of claim 1, further comprising:
at least a medicinally effective amount of one compound from the group consisting of Magnesium Gluconate, Vitamin B6 [Pyridoxal-phosphate], and Vitamin D3 [Cholecalciferol].

3. The composition of claim 1, further comprising:
at least a medicinally effective amount of two compounds from the group consisting of Magnesium Gluconate, Vitamin B6 [Pyridoxal-phosphate], and Vitamin D3 [Cholecalciferol].

4. The composition of claim 1, further comprising:
a medicinally effective amount of Magnesium gluconate,
an amount of Vitamin B6 [Pyridoxal-phosphate], and
an amount of Vitamin D3 [Cholecalciferol].

5. The composition of claim 1 wherein the medicinally effective amounts of the combination of DL-Phenylalanine, L-Tyrosine and L-Glutamine includes each between 0.02 mg per pound body mass per day of the mammal and 240 mg per pound body mass of the mammal.

6. The composition of claim 1 wherein the medicinally effective amounts of the combination of DL-Phenylalanine, L-Tyrosine and L-Glutamine includes between 0.02 mg per pound body mass per day of the human and 180 mg per pound body mass per day of the human.

7. The composition of claim 4 wherein the medicinally effective amounts of compounds are:
a. for Magnesium Gluconate between 0.02 mg per pound body mass per day of the mammal and 240 mg per pound body mass per day of the mammal.
b. for Vitamin B6 [Pyridoxal-phosphate] between 0.02 mg per pound body mass per day of the mammal and 240 mg per pound of body mass per day of the living mammal, and
c. for Vitamin D3 [Cholecalciferol F] between 0.0002 mg per pound body mass per day of the mammal and 1 mg per pound of body mass per day of the mammal.

8. The composition of claim 4 wherein the medicinally effective amounts of compounds are:
a. for Magnesium Gluconate between 0.02 mg per pound body mass of the human and 240 mg per pound body mass per day of the human,
b. for Vitamin B6 [Pyridoxal-phosphate] between 0.02 mg per pound body mass per day of the human and 240 mg per pound body mass per day of the human, and
c. for Vitamin D3 [Cholecalciferol F] between 0.02 microgram per pound body mass per day of the human and 240 microgram per pound body mass per day of the human.

9. The composition of claim 1 wherein at least one of DL-Phenylalanine, L-Tyrosine and L-Glutamine are in the form of at least one of N-Acetyl-DL-Phenylalanine, N-Acetyl-L-Tyrosine, and N-Acetyl-L-Glutamine.

10. The composition of claim 1 wherein at least one of DL-Phenylalanine, L-Tyrosine and L-Glutamine are in the form of one or more peptides.

11. A method of achieving greater balance in an imbalanced sympathetic nervous system, comprising the steps of:
a. providing a composition, comprising:
i. an medicinally effective amount of the combination of:
(a). DL-Phenylalanine,
(b). L-Tyrosine, and
(c). L-Glutamine, and
ii. no discernibly effective amount of each of:
(a). St. John's Wort,
(b). chromium picolinate,
(c). digestive enzymes such as pepsin and bromelain,
(d). furanones,
(e). branched chain amino acids, and
(f). peptides containing more than three peptide forms of amino acids different from those of DL-Phenylalanine, L-Tyrosine and L-Glutamine; and
b. taking a recommended quantity of the composition over a recommended time frame to reduce symptoms.

12. The method of claim 11, further comprising the steps of:
c. taking a recommended quantity of the composition over a recommended time frame to maintain a reduced level of symptoms.
